(19) United States
(12) Patent Application Publication
(10) Pub. No.: US 2009/0068266 A1
(43) Pub. Date: Mar. 12, 2009

(54) SIROLIMUS HAVING SPECIFIC PARTICLE SIZE AND PHARMACEUTICAL COMPOSITIONS THEREOF

(76) Inventors: Praveen RAHEJA, New Delhi (IN); Atul KAUSHIK, Ghaziabad (IN); Rajesh GANDHI, Gurgaon (IN); Romi Barat SINGH, Varanasi (IN); Rajeev Shanker MATHUR, Gurgaon (IN)

Correspondence Address: Jayadeep R. Deshmukh, Esq.;Ranbaxy Inc., Suite 2100, 600 College Road East, Princeton, NJ, 08540, US

(21) Appl. No.: 12/125,225

(22) Filed: May 22, 2008

(30) Foreign Application Priority Data
Sep. 11, 2007 (IN) 1922/DEL/2007

(51) Int. Cl. A61K 9/28; C07D 498/18; A61K 31/4355; A61K 9/14

(52) U.S. Cl. 424/474; 540/456; 514/291; 424/489; 424/490

(57) ABSTRACT

Sirolimus particles having d₉₀ value of from about 2μ to about 10μ have been developed. Further, pharmaceutical composition comprising sirolimus particles having d₉₀ value of from about 2μ to about 10μ have also been developed.

### FIELD OF THE INVENTION

The present invention relates to sirolimus particles having d₉₀ value of from about 2μ to about 10μ. It further relates to a pharmaceutical composition comprising said particles.

### BACKGROUND OF THE INVENTION

Rapamycin is a macrolide antibiotic produced by Streptomyces hygroscopicus which was first found to have antifungal properties. It adversely affects the growth of fungi such as Candida albicans and Microsporum gypseum. Rapamycin, its preparation and its antibiotic activity were described in U.S. Pat. No. 3,929,992. In Martel, R. R. et al. 1977 reported immunosuppressive properties of rapamycin against experimental allergic encephalitis and adjuvant arthritis in the Canadian Journal of Physiological Pharmacology, 55, (1977) 48-51. In 1989, Calne, R. Y. et al. in Lancet, no. 2, (1989), p. 227 and Morris, R. E. and Meiser, B. M. in Medicinal Science Research, No. 17, 1989, p. 609-10, separately reported the effectiveness of rapamycin in inhibiting rejection in vivo in allograft transplantation. U.S. Pat. No. 5,100,899 discloses the use of Rapamycin to inhibit transplantation rejection in mammals.

Its poor oil and water solubility, poses a significant problems in formulating the drug into suitable dosage form. In addition, it has been reported that compositions of Sirolimus with conventional excipients show unpredictable dissolution rates, irregular bioavailability profiles, as well stability problems. Currently, Sirolimus is available in two dosage forms namely tablet and oral solution.

U.S. Pat. Nos. 5,989,591 and 5,985,325 disclose a solid dosage unit of rapamycin comprising a core, which is over coated with rapamycin, and a sugar coat containing one or more surface modifying agents, one or more sugars and optionally one or more binders.

U.S. Pat. No. 5,145,684 discloses a nanoparticulate composition comprising particles consisting of a poorly soluble drug having adsorbed onto the surface thereof a non-crosslinked surface stabilizer wherein effective average particle size of drug substance is less than about 400 nm.

Nanonization of poorly soluble drug is a complex process and requires additional step during manufacturing. Moreover, nanonization increases the surface area available for dissolution; however, it also increases the change in free energy of the system when exposed to an aqueous solution. This results in particle aggregation and decreases the dissolution rate. Also, very fine particles are difficult to handle due to static charge that develops on particle surface during processing.

### SUMMARY OF THE INVENTION

Now, we have found that sirolimus particles having d₉₀ value of from about 2μ to about 10μ provides the desired in vitro and in vivo profile.

Hence, according to one of the aspects, there is provided sirolimus particles having d₉₀ value of from about 2μ to about 10μ.

In another aspect, there is provided sirolimus particles having d₉₀ value of from about 2μ to about 10μ and d₅₀ value of from about 0.5μ to about 4μ

In another aspect, there is provided a process of preparation of sirolimus particles comprising the step of micronising coarser sirolimus particle using milling to obtain sirolimus particles having d₉₀ value of from about 2μ to about 10μ.

In another aspect, there is provided a pharmaceutical composition comprising sirolimus particles having d₉₀ value of from about 2μ to about 10μ.

In another aspect, there is provided a pharmaceutical composition of sirolimus comprising a dispersion of sirolimus particles and one or more pharmaceutically acceptable excipients wherein said sirolimus particles have d₉₀ value of from about 2μ to about 10μ.

In another aspect, there is provided a process of preparing a pharmaceutical composition of sirolimus, comprising the steps of:
a) Dissolving or dispersing one or more pharmaceutically acceptable ingredients in a vehicle; and
b) Dispersing sirolimus particles in the dispersion/solution of step a);
   wherein said sirolimus particles have d₉₀ value of from about 2μ to about 10μ.

In another aspect, there is provided a process of preparing a pharmaceutical composition of sirolimus particles, comprising the steps of:
a) Dissolving or dispersing one or more pharmaceutically acceptable excipients in a vehicle;
b) Dispersing sirolimus particles in the dispersion/solution of step a); and
c) Processing the dispersion of step b) into suitable pharmaceutical composition;
   wherein said sirolimus particles have d₉₀ value of from about 2μ to about 10μ.

In another aspect, there is provided a pharmaceutical composition comprising
a) a dispersion of sirolimus particles and one or more pharmaceutically acceptable excipients in a vehicle;
b) an inert core coated with said dispersion; and
c) optionally, coating said drug coated cores to obtain the desired dosage form;
   wherein said sirolimus particles have d₉₀ value of from about 2μ to about 10μ.

In another aspect, there is provided a method of treatment of organ or tissue transplant rejection, autoimmune disease, inflammatory conditions, or multi-drug resistance, the method comprising: orally administering to a subject a pharmaceutical composition comprising sirolimus particles having d₉₀ value of from about 2μ to about 10.

### DETAILED DESCRIPTION OF THE INVENTION

Sirolimus” as employed herein is intended to include amorphous or crystalline form of the drug. The crystalline form may include polymorph form I or II or a mixture thereof.

The known particle size analysis methods can be used for determining the particle size, for example particle size measurement using light, like light-scattering methods, in particular Malvern Mastersizer.

The term “d₉₀ value” means at east 90% of sirolimus particles have volume diameter in the specified range when measured by a light scattering method for example Malvern Mastersizer.

The term “d₅₀ value” means at least 50% of sirolimus particles have volume diameter in the specified range when measured by a light scattering method for example Malvern Mastersizer

Micronization may be carried out using dry milling technique. Various conventional mills available for dry milling are ball mill, an attritor mill, a vibratory mill, air jet mill and media mills such as a sand mill and a bead mill. The milling may be carried out using the sirolimus alone or with other pharmaceutically acceptable excipients. Also, supercritical fluid technique may be utilized for particle size reduction. The desired particle size may also be obtained by modifying the reaction conditions during the manufacturing of Sirolimus API.

“Pharmaceutical composition” as used herein includes both liquid and solid dosage forms such as solution, suspension, tablet, capsule, granules and pills.

Pharmaceutical composition may be in the form of tablet comprising an inert core and coating of sirolimus dispersion.

“Inert core” as used herein includes inert tablet core or inert beads or spheres.

Inert tablet core may be further coated with sugar dispersion/solution. Sugar coating may be in the form of seal coating, sub coating, syrup coating and the like.

Seal coating is used to prevent moisture penetration into the tablet core and thus prevents the tablet core from disintegrating during the over coating process. Seal coating may comprise shellac, oleic acid, propylene glycol, talc, polyethylene glycol or mixture thereof.

Sub coating as used herein is used to round the edges and build up the tablet size. Sub coating in addition to sugar may comprise other excipients selected from the group consisting of starch, talc, calcium carbonate, calcium sulfate or mixture thereof.

Other than sugar coating, tablet may further comprise film coating such as functional or non functional layer. The coating may be selected from amongst one or more of those suitable coating materials known in the art. Coating may be performed by applying one or more film forming polymers, with or without other pharmaceutically inert excipients, as a solution/suspension.

Coating is done using any conventional coating technique known in the art, such as spray coating in a conventional coating pan or fluidized bed processor; or dip coating.

The term “pharmaceutically acceptable excipients” as used herein include surface modifiers, sugars, binders, diluents, lubricant/glidant, disintegrating agent, antioxidants and coloring agents. These agents may be present in the core or coating or both.

The term “surface modifiers” as used herein means agents which are used to disperse the drug in a particular vehicle and also enhance wetting properties of the drug. Such excipients include various polymers, low molecular weight oligomers, natural products and surfactants. Representative examples include gelatin, casein, lecithin (phosphatides), gum acacia, cholesterol, tragacanth, stearic acid, benzalkonium chloride, calcium stearate, colloidal silicon dioxide, phosphates, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, and polyvinylpyrrolidone. Surfactants include both non-ionic and ionic (cationic, anionic and zwitterionic) surfactants suitable for use in pharmaceutical dosage forms. These include polyethoxylated fatty acids and its derivatives, for example polyethylene glycol 400 distearate, polyethylene glycol-20 dioleate, polyethylene glycol 4-150 mono dilaurate, polyethylene glycol-20 glyceryl stearate; alcohol-oil transesterification products, for example polyethylene glycol-6 corn oil; polyglycerized fatty acids, for example polyglyceryl-6 pentaoleate; propylene glycol fatty acid esters, for example propylene glycol monocaprylate; mono and diglycerides for example glyceryl ricinoleate; fatty acids and their esters such as glyceryl monooleate, sterol and sterol derivatives; sorbitan fatty acid esters and its derivatives, for example polyethylene glycol-20 sorbitan monooleate, sorbitan monolaurate; polyethylene glycol alkyl ether or phenols, for example polyethylene glycol-20 cetyl ether, polyethylene glycol-10-100 nonyl phenol; sugar esters, for example sucrose monopalmitate; polyoxyethylene-polyoxypropylene block copolymers known as “poloxamer”, such as Poloxamer 237, 338 and 407; ionic surfactants, for example sodium caproate, sodium glycocholate, soy lecithin, sodium stearyl fumarate, propylene glycol alginate, octyl sulfosuccinate disodium, palmitoyl camitine. These may be used from the same category or a combination of surfactant with low molecular weight oligomers/natural products.

Sugars may be used to prepare sugar barrier coat or over coat or drug coat wherein the drug coat comprises dispersion of the sirolimus and sugars or one or more pharmaceutical acceptable excipients. Sugar may include lactose, mannitol, sorbitol, sucrose and mixtures thereof.

Specific examples of “binders” include methyl cellulose, hydroxypropyl cellulose, hydroxylpropyl methycellulose, polyvinylpyrrolidone, gelatin, gum Arabic, ethyl cellulose, polyvinyl alcohol, pullulan, pregelatinized starch, agar, tragacanth, carboxymethyl cellulose, sodium alginate, propylene glycol, microcrystalline cellulose or mixtures thereof.

The term “diluents” as used herein includes calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, sucrose and mixtures thereof.

Specific examples of lubricants/glidants include colloidal silicon dioxide, stearic acid, magnesium stearate, calcium stearate, talc, hydrogenated castor oil, and mixtures thereof.

Disintegrating agent for the present invention may be selected from starches or modified starches such as starch, modified starch, croscarmellose sodium, crospovidone and sodium starch glycolate.

The composition may further comprise antioxidant, to protect the drug from oxidative degradation. Antioxidants may be selected from group consisting of ascorbic acid, sodium pyrosulphite, glutathion or sorbic acid, tocopherol and the like, in particular tocopherol E-acetate.

Coloring agent may be selected from FDA approved colorants and the examples are Iron oxide, Opalux yellow, Lake of Tartrazine, Allura red, Lake of Quinoline yellow, Lake of Erythrosine.

The vehicle used to prepare the dispersion may be selected from water or its mixture with other organic solvent such as ethanol, methanol, isopropyl alcohol and ether. According to one of the embodiment, sirolimus particles are prepared by micronising sirolimus coarser particles by dry milling technique to obtain a desired particle size range.

According to another embodiment, sirolimus particles are prepared by micronising sirolimus coarser particles by supercritical fluid technology to obtain a desired particle size range.

According to another embodiment, there is provided a process for the preparation of liquid pharmaceutical composition of sirolimus by dissolving/dispersing sirolimus particles of the desired particle size along with other pharmaceutically acceptable excipients into a suitable vehicle to obtain dispersion or solution of sirolimus.

According to another embodiment, process for the preparation of solution comprises the following steps
i) Compressing pharmaceutically acceptable excipients to obtain inert tablet core;
ii) Dissolving/dispersing sirolimus particles of the desired particle size along with other pharmaceutically acceptable excipients into a suitable vehicle;
iii) Coating dispersion of step ii) onto the inert tablet core of step i);
iv) Optionally, coating the drug coated core of step iii).

The invention is further illustrated by the following examples but they should not be construed as limiting the scope of this invention in any way.

### EXAMPLES

### Example 1

Coarser sirolimus particles were micronized to obtain desired particle size and particle size distribution is given below.

d₁₀—0.1μ
d₅₀—0.9μ
d₉₀—4.2μ

### Example 2

Sirolimus tablet were prepared by using sirolimus particles of Example 1

| Ingredient | Qty/tab (mg) |
|---|---|
| Inert core tablets | |
| Lactose | 129.00 |
| Polyethylene glycol-6000 | 15.00 |
| Talc | 3.00 |
| Magnesium stearate | 3.00 |
| Seal coating | |
| Pharmaceutical glaze | 9.00 |
| (50% shellac solution) | |
| Talc | q.s |
| Absolute alcohol | q.s to make 25% solution |
| Sub Coating | |
| Sub Coat* | 38.60 |
| Talc | q.s |
| water | q.s |
| Sugar barrier coat | |
| Sucrose | 7.95 |
| Hydroxypropyl | 0.05 |
| methylcellulose | |
| Microcrystalline cellulose | 2.00 |
| Purified water | qs |
| Drug layering | |
| Sirolimus (obtained from | 2.00 |
| example 1) | |
| Poloxamer-407 | 1.00 |
| Hydroxypropyl | 0.50 |
| methylcellulose | |
| Microcrystalline cellulose | 2.00 |
| Tocopherol E-acetate | 0.50 |
| Sucrose | 94.00 |
| Purified water | q.s |
| Over coat | |
| Sucrose | 33.00 |
| Hydroxypropyl | 0.17 |
| methylcellulose | |
| Tocopherol E-acetate | 0.50 |
| Water | qs |
| Color Coat | |
| Opalux yellow | 20.00 |
| Water | qs |
| Polishing | |
| Carnauba wax | 1.00 |
| Methanol | qs |

*Sub Coat contains Sucrose-65%, Calcium Sulfate-22%, MCC-8%, Macrogol/PEG-20000-2% and Titanium dioxide-2%.
**contains DL-alpha tocopherol acetate (50%) starch, fish gelatin, sugar, Silicon-di-oxide (E 551)

### Procedure:

### A. Preparation of Inert Core

i) Lactose, polyethylene glycol, talc and magnesium stearate were blended together and compressed into a suitable tablet;

### B. Seal Coating

i) Pharmaceutical glaze was diluted to 25% w/w solution using absolute alcohol;
ii) Inert tablets of step A were coated with solution of step i) and during the coating process talc was intermittently sprinkled to prevent sticking of the tablets;

### C. Sub Coating

i) Sub coat was dispersed in water to obtain a 70% w/w of sub coat suspension;
ii) The suspension of step i) was used to coat the coated tablets of step B and during the coating process talc was intermittently sprinkled to prevent sticking of the tablets;

### D. Sugar Barrier Coat

i) Sucrose, microcrystalline cellulose and hydroxypropyl methylcellulose were dispersed in water;
ii) Dispersion of step i) was coated over the coated tablet of step C;

### E. Drug Layering

i) Poloxamer 407 was dissolved in water;
ii) Hydroxypropyl methylcellulose was dissolved in solution of step i);
iii) Sucrose was added in solution of step ii) under stirring;
iv) Microcrystalline cellulose was dispersed in syrup of step iii) under stirring;
v) Sirolimus was dispersed in syrup of step iv) under stirring;
vi) Tocopherol was dispersed in dispersion of step v) under stirring;
vii) The resulting dispersion was coated onto the sugar barrier coated tablet of step D;

### F. Over Coat

i) Sucrose, tocopherol and HPMC were dispersed in water;
ii) Dispersion of step i) was coated over the coated tablet of step E;

### G. Color Coat

i) Opalux yellow was dispersed in water;
ii) Dispersion of step i) was coated onto the over coated tablet of step F;

### H. Polishing

i) Carnauba wax was dispersed in methanol;
ii) Dispersion of step i) was coated over the color coated tablet of step G.

### Bioequivalence Study

Bioavailability study of the Sirolimus tablet (2 mg) tablet of example 2 was carried out on healthy male volunteers (n=8) taking Rapamune® (2 mg) produced by Wyeth Pharmaceuticals as the reference, the results of which are represented in Table 1 and 2. The objective of this study was to show that a formulation of Example 2 provides an activity and safety profile that is similar to one obtained with an equivalent product in the market.

Single dose (2 mg) two way crossover, and open randomized study was designed as, two treatment, two period, two sequence was used for comparative bioavailability of sirolimus tablet of Example 2 and Rapamune® tablet (2 mg) of Wyeth Pharmaceuticals, under fasting and fed conditions.

TABLE 1
Comparative pharmacokinetic parameters for the sirolimus
(Example 2) and Rapamune ® tablet under fed conditions.

| | | AUC₀₋ₜ | AUC_{0-∞} |
|---|---|---|---|
| N = 7 | Cₘₐₓ (ng/ml) | (ng · h/ml) | (ng · h/ml) |
| Sirolimus tablet | 17.4964 | 355.6890 | 420.9476 |
| (Test) | CV % 30.4 | CV % 46.6 | CV % 41.5 |
| Sirolimus tablet | 16.7475 | 348.2036 | 434.1065 |
| (Ref.) | CV % 22.9 | CV % 32.4 | CV % 35.5 |
| Test/Ref. % | 104.90 | 102.56 | 96.68 |
| (90% | (93.29-117.95)% | (80.77-130.24)% | (79.92-116.95)% |
| confidence | | | |
| interval) | | | |

TABLE 2
Comparative pharmacokinetic parameters for the sirolimus
(Example 2) and Rapamune ® tablet under fasting conditions.

| | | AUC₀₋ₜ | AUC_{0-∞} |
|---|---|---|---|
| | Cₘₐₓ (ng/ml) | (ng · h/ml) | (ng · h/ml) |
| Sirolimus tablet | 8.9517 | 301.5530 | 381.9564 |
| (Test) | CV % 21.6 | CV % 32.1 | CV % 32.5 |
| Sirolimus tablet | 9.2593 | 290.3494 | 380.4703 |
| (Ref.) | CV % 37.1 | CV % 25.5 | CV % 21.8 |
| Test/Ref. % | 97.67 | 103.66 | 100.81 |
| (90% | (81.88-116.50)% | (86.45-124.29)% | (81.67-124.42)% |
| confidence | | | |
| interval) | | | |

AUC_{0-∞} for sirolimus tablet was within 80-125% (at 90% Confidence Interval) as shown in Table 1 and 2. The results show that sirolimus 2 mg tablets prepared as per the examples described herein have bioavailability comparable to the reference product, Rapamune® tablet 2 mg of Wyeth Pharmaceuticals, USA.

### Example 3

Sirolimus particles having particle size distribution as given below were obtained.

### Batch 1

d₁₀—0.1μ
d₅₀—0.9μ
d₉₀—4.2μ

### Batch 2

d₅₀—2.060μ
d₉₀—4.919μ

### Batch 3

d₅₀—2.321μ
d₉₀—5.974μ

### Batch 4

d₅₀—1.877μ
d₉₀—6.430μ
d₁₀—0.678μ

### Batch 5

d₅₀—2.488μ
d₉₀—6.775μ
d₁₀—0.865μ

### Batch 6

d₅₀—1.977μ
d₉₀—4.958μ
d₁₀—0.784μ

### Example 4

| Ingredient | Qty/tab (mg) |
|---|---|
| Inert core tablets | |
| Lactose | 129.00 |
| Polyethylene glycol-6000 | 15.00 |
| Talc | 3.00 |
| Magnesium stearate | 3.00 |
| Seal coating | |
| Pharmaceutical glaze | 3.50 |
| (50% shellac solution) | |
| Talc | 1.00 |
| Absolute alcohol | q.s to make 25% |
| | solution |
| Sub Coating | |
| Sub Coat* | 38.00 |
| Talc | 0.50 |
| water | q.s |
| Sugar barrier coat | |
| Sucrose | 8.00 |
| Microcrystalline cellulose | 2.00 |
| Purified water | qs |
| Drug layering | |
| Sirolimus | 2.04 |
| Poloxamer-407 | 1.00 |
| Hydroxypropyl methylcellulose | 0.20 |
| Microcrystalline cellulose | 0.20 |
| Vitamin E | 0.25 |
| Sucrose | 96.31 |
| Purified water | q.s |
| Over coat | |
| Sucrose | 36.00 |
| Water | qs |
| Color Coat | |
| Opalux yellow | 2.40 |
| Sucrose | 18.47 |
| Hydroxypropyl methylcellulose | 0.10 |
| Water | qs |
| Polishing | |
| Carnauba wax | 0.03 |
| Methanol | qs |

*Sub Coat contains Sucrose-65%, Calcium Sulfate-22%, MCC-8%, Macrogol/PEG-20000-2% and Titanium dioxide-2%.

### Procedure:

### A. Preparation of Inert Core

i) Lactose, polyethylene glycol, talc and magnesium stearate were blended together and compressed into a suitable tablet;

### B. Seal Coating

i) Pharmaceutical glaze was diluted to 25% w/w solution using absolute alcohol
ii) Inert tablets of step A were coated with solution of step i) and during the coating process talc was intermittently sprinkled to prevent sticking of the tablets;

### C. Sub Coating

i) Sub coat was dispersed in water to obtain a 70% w/w of sub coat suspension;
ii) The suspension of step i) was used to coat the coated tablets of step B and during the coating process talc was intermittently sprinkled to prevent sticking of the tablets;

### D. Sugar Barrier Coat

i) Sucrose, and MCC were dispersed in water;
ii) Dispersion of step i) was coated over the coated tablet of step C;

### E. Drug Layering

i) Poloxamer 407 was dissolved in part of water;
ii) Sirolimus was dispersed in solution of step i) under stirring;
iii) Hydroxypropyl methylcellulose was dissolved in another part of water;
iv) Vitamin E was loaded over sucrose using low shear mixture;
v) Vitamin E loaded sucrose was dispersed in solution of step iii);
vi) Dispersion of step ii) was added into dispersion of step v);
vii) Microcrystalline cellulose was dispersed in dispersion of step vi) under stirring;
(vii) The resulting dispersion (d₉₀—5.46 and d₅₀—2.12) was coated onto the sugar barrier coated tablet of step D;

### F. Over Coat

i) Sucrose was dispersed in water;
ii) Dispersion of step i) was coated over the coated tablet of step E;

### G. Color Coat

i) HPMC and sucrose were dissolved in water;
ii) Opalux yellow was dispersed in solution of step i);
iii) Dispersion of step ii) was coated onto the over coated tablet of step F;

### H. Polishing

i) Carnauba wax was dispersed in methanol
ii) Dispersion of step i) was coated over the color coated tablet of step G.

### Example 5

| Ingredient | Qty/tab (mg) |
|---|---|
| Inert core tablets | |
| Lactose | 129.00 |
| Polyethylene glycol-6000 | 15.00 |
| Talc | 3.00 |
| Magnesium stearate | 3.00 |
| Seal coating | |
| Pharmaceutical glaze | 3.50 |
| (50% shellac solution) | |
| Talc | 1.00 |
| Absolute alcohol | q.s to make 25% |
| | solution |
| Sub Coating | |
| Sub Coat* | 38.00 |
| Talc | 0.50 |
| water | q.s |
| Sugar barrier coat | |
| Sucrose | 7.95 |
| Microcrystalline cellulose | 2.00 |
| HPMC | 0.05 |
| Purified water | qs |
| Drug layering | |
| Sirolimus | 2.04 |
| Poloxamer-407 | 1.00 |
| Hydroxypropyl methylcellulose | 0.50 |
| Microcrystalline cellulose | 2.00 |
| Vitamin E | 0.25 |
| Sucrose | 94.25 |
| Purified water | q.s |
| Over coat | |
| Sucrose | 35.82 |
| HPMC-E5 | 0.18 |
| Water | qs |
| Color Coat | |
| Opalux yellow | 2.40 |
| Sucrose | 17.5 |
| Hydroxypropyl methylcellulose | 0.10 |
| Water | qs |
| Polishing | |
| Carnauba wax | 1.0 |
| Methanol | qs |

*Sub Coat contains Sucrose-65%, Calcium Sulfate-22%, MCC-8%, Macrogol/PEG-20000-2% and Titanium dioxide-2%.

### Procedure:

### A. Preparation of Inert Core

i) Lactose, polyethylene glycol, talc and magnesium stearate were blended together and compressed into a suitable tablet;

### B. Seal Coating

i) Pharmaceutical glaze was diluted to 25% w/w solution using absolute alcohol;
ii) Inert tablets of step A were coated with solution of step i) and during the coating process talc was intermittently sprinkled to prevent sticking of the tablets;

### C. Sub Coating

i) Sub coat was dispersed in water to obtain a 70% w/w of sub coat suspension;
ii) The suspension of step i) was used to coat the coated tablets of step B and during the coating process talc was intermittently sprinkled to prevent sticking of the tablets;

### D. Sugar Barrier Coat

i) Sucrose, HPMC and MCC were dispersed in water;
ii) Dispersion of step i) was coated over the coated tablet of step C;

### E. Drug Layering

i) Poloxamer 407 was dissolved in part of water;
ii) Sirolimus was dispersed in solution of step i) under stirring;
iii) Hydroxypropyl methylcellulose was dissolved in another part of water;
iv) Vitamin E was loaded over sucrose using low shear mixture;
v) Vitamin E loaded sucrose was dispersed in solution of step iii);
vi) Dispersion of step ii) was added into dispersion of step v);
vii) Microcrystalline cellulose was dispersed in dispersion of step vi) under stirring;
viii) The resulting dispersion was coated onto the sugar barrier coated tablet of step D;

### F. Over Coat

i) Sucrose and HPMC were dispersed in water;
ii) Dispersion of step i) was coated over the coated tablet of step E;

### G. Color Coat

i) HPMC and sucrose were dissolved in water;
ii) Opalux yellow was dispersed in solution of step i);
iii) Dispersion of step ii) was coated onto the over coated tablet of step F;

### H. Polishing

i) Carnauba wax was dispersed in methanol;
ii) Dispersion of step i) was coated over the color coated tablet of step G.

We claim:

1. Sirolimus particles having d₉₀ value of from about 2μ to about 1μ.

2. The sirolimus particles according to claim 1 wherein the sirolimus particles have d₅₀ value of from about 0.5 to about 4μ.

3. The sirolimus particles according to claim 1 wherein the sirolimus particles are prepared using milling technique.

4. The pharmaceutical composition of sirolimus particles of claim 1 wherein composition is a liquid dosage form.

5. The pharmaceutical composition of sirolimus particles of claim 1 wherein composition is a solid dosage form.

6. The pharmaceutical composition according to claim 5 wherein the solid dosage form comprises a dispersion of sirolimus particles coated onto an inert core.

7. The pharmaceutical composition according to claim 6 wherein the coated tablet is prepared by a process comprising the steps of:
a) dispersing sirolimus particles and one or more pharmaceutically acceptable excipients in a vehicle;
b) coating an inert core with said dispersion; and
c) optionally, coating said drug coated cores to obtain the desired solid dosage form.

8. The pharmaceutical composition according to claim 7 wherein the pharmaceutically acceptable excipients are selected from the group consisting of surface modifiers, sugars, binders, diluents, lubricant/glidant, disintegrating agent, antioxidants and coloring agents.
